(19) United States
(12) Patent Application Publication
(10) Pub. No.: US 2019/0307983 A1
(43) Pub. Date: Oct. 10, 2019

(54) STANDALONE HANDHELD WELLNESS DEVICE

(71) Applicant: ENSO CO., San Francisco, CA (US)

(72) Inventor: Rebecca L. GOLDMAN, San Francisco, CA (US)

(21) Appl. No.: 16/450,207

(22) Filed: Jun. 24, 2019

Related U.S. Application Data
(63) Continuation of application No. PCT/US2017/067652, filed on Dec. 20, 2017.
(60) Provisional application No. 62/438,975, filed on Dec. 23, 2016.
(60) Provisional application No. 62/477,142, filed on Mar. 27, 2017.

(51) Int. Cl. A61M 21/02; A61B 5/0205; A61B 5/16; A61H 23/02

(52) U.S. Cl. CPC A61M 21/02; A61B 5/0205; A61M 2021/0022; A61H 23/02; A61B 5/16

(57) ABSTRACT

The disclosed embodiments include a standalone handheld wellness device. The wellness device can have a housing with dimensions for being held in a palm of a user's hand. The housing can include an input mechanism that detects signals indicative of the user's physiological activity obtained from the user while the user is holding the wellness device in the palm of the user's hand, and processing circuitry that generates physiological information based on the obtained signals indicative of the physiological activity. The physiological information may indicate a pattern of a user's bodily function while the wellness device is held in the palm of the user's hand. The housing can also include an output mechanism that generates a physically perceptible output based on the physiological information such that the physically perceptible output is a physical rendering of the pattern of the user's bodily function.

### CROSS-REFERENCE TO RELATED APPLICATIONS

This application claims to the benefit of U.S. Provisional Patent Application No. 62/438,975 titled “Standalone Handheld Wellness Device” and filed on Dec. 23, 2016, and of U.S. Provisional Patent Application No. 62/477,142 titled “Wireless Bonding Between a Handheld Wellness Device and a Computing Device” and filed on Mar. 27, 2017, which are each incorporated by reference herein in their entireties.

### TECHNICAL FIELD

The disclosed teachings relate to a standalone handheld wellness device. More particularly, the standalone handheld wellness device can generate physically perceptible outputs based on physiological information such that a user of the standalone handheld wellness device can physically perceive physiological activity and/or be induced to change the user's physiological activity based on the outputs.

### BACKGROUND

There are millions of people that often live trapped in their own thoughts—stressed and disconnected from their bodies and from other people. The stress and social isolation causes undesired physical symptoms such as muscle tension, rapid and/or shallow breathing, increased heart rate, and general discomfort. Chronic stress can contribute to long term physical problems including an increased risk of heart attacks or depression. In some cases, people turn to wellness activities (e.g., yoga) to learn how to control or reduce their stress. Wellness generally refers to a healthy balance of the mind, body, and spirit that results in an overall feeling of well-being.

People also turn to using activity trackers to gain awareness of physiological activity that is not readily perceptible and/or burdensome to monitor manually. In some instances, activity trackers are consumer products that people use as part of a wellness program to improve fitness and overall health by monitoring physiological activity. For example, a person can wear an activity tracker (e.g., FITBIT tracker) to monitor the person's physiological activity. The activity tracker may be a wireless-enabled wearable technology device that measures physiological activity to determine the number of steps walked, heart rate, quality of sleep, steps climbed, and other personal metrics involved in fitness and health. The activity tracker can generate information that can be rendered on a display of a computing device. For example, a personal computer can display charts and graphs based on the person's activity as captured by the activity tracker.

In more severe cases, persons turn to medical wellness programs. A medical wellness program may include the use of a biofeedback system that trains a person to voluntarily change the person's physiological activity based on biofeedback (i.e., responsive to the person's monitored physiological activity). For example, a biofeedback system can include electrodes attached to a person's body and connected to a computer. The physiological activity detected by the electrodes generates signals communicated to a computer. The signals are processed to generate output as feedback rendered on a computer display, which may induce a change in physiological activity, thinking, and/or emotions. Therapists can use biofeedback when treating anxiety, chronic pain, headaches, and the like. Although beneficial, these biofeedback systems are cost-prohibitive for everyday consumers, bulky, cumbersome, and complex such that a skilled professional is required to use such systems. Accordingly, a need exists for user-friendly and cost-effective wellness tools that can help people combat stress and feelings of being disconnected from their bodies and from other people.

### SUMMARY

The embodiments disclosed herein include a standalone handheld wellness device with a housing dimensioned to enable holding the wellness device in a palm of a user's hand. The housing can include an input mechanism operable to detect signals indicative of the user's physiological activity obtained from the user while the user is holding the wellness device in the palm of the user's hand, and processing circuitry operable to generate physiological information based on the obtained signals indicative of the physiological activity. The physiological information may translate a pattern of the user's bodily function into a more easily perceived form (e.g., with color, sound, vibration) occurring while the user is holding the wellness device in the palm of the user's hand. Hence, the housing can also include an output mechanism operable to generate a physically perceptible output based on the physiological information such that the physically perceptible output is a physical rendering of the pattern of the user's bodily function or derived therefrom (e.g. a relax mode output can be at −20% of an actual rate).

In some embodiments, the bodily function is an autonomic bodily function including any of a heart rate, a heart rate variability, a blood oxygenation level, digestion activity, a respiratory rate, a pupillary response, urination activity, or sexual arousal. In some embodiments, the bodily function is a heartbeat of the user.

In some embodiments, the bodily function is a heartbeat of the user and the physically perceptible output includes at least one of a rate or a magnitude of the user's heartbeat.

In some embodiments, the physically perceptible output is generated in real-time or near real-time. In some embodiments, the physically perceptible output is synchronized with the user's bodily function. In some embodiments, the physically perceptible output tracks the pattern of the bodily function. In some embodiments, the physically perceptible output has at least one of a rate or magnitude.

In some embodiments, the standalone computing device further includes a memory operable to store the physiological information such that the physically perceptible output is based on the physiological information retrieved from the memory. In some embodiments, the standalone computing device further includes a memory operable to store the physiological information such that the physically perceptible output is rendered asynchronously from the stored physiological information.

In some embodiments, the input mechanism includes a sensor configured to monitor a heartbeat of the user in real-time while the user is holding the standalone computing device in the palm of the user's hand.

In some embodiments, the output mechanism includes a vibration motor configured to vibrate the standalone computing device in accordance with the physiological information.

In some embodiments, the output mechanism includes a light source configured to illuminate at least a portion of the standalone computing device in accordance with the physiological information. In some embodiments, the light source generates an illumination pattern that tracks the pattern of the bodily function. In some embodiments, the illumination pattern includes multiple colors. In some embodiments, each of the multiple colors of the illumination pattern indicate a different range of the physiological information including at least one of a rate or magnitude of the pattern of the user's bodily function. In some embodiments, the illumination pattern changes colors in real-time or near-real time with a change of least one of a rate or magnitude of the pattern of the user's bodily function as indicated in the physiological information.

In some embodiments, the standalone computing device is a first standalone computing device that further includes a wireless transmitter configured to transmit the physiological information to a second standalone computing device and causes the second standalone computing device to render physiological perceptible output based on the physiological information.

In some embodiments, the standalone computing device is a first standalone computing device that further includes a wireless receiver configured to receive physiological information from a second standalone computing device and causes the first standalone computing device to render physiological perceptible output based on the received physiological information.

In some embodiments, the standalone computing device further includes a wireless transceiver configured to exchange physiological information with another standalone computing device such that each standalone computing device renders physiological perceptible output based on physiological information of the other standalone computing device.

In some embodiments, the output mechanism includes multiple components operable to generate multisensory physically perceptible output based on the physiological information.

In some embodiments, the standalone computing device includes a memory operable to store the physiological information recorded for a period of time as selected by the user.

In some embodiments, the standalone computing device further includes an accelerometer configured to identify a user input based on a movement of the standalone computing device.

In some embodiments, the physiological activity is indicative of a breathing pattern of the user.

In some embodiments, the housing is shaped as a river stone.

Embodiments also include a self-contained handheld device that has a body with a shape that resembles a river stone. The body can include a sensor configured to collect signals indicative of a user's heartbeat while the self-contained handheld device is positioned in contact with the user's skin on the palm of the user's hand, and a processor configured to collect and/or process data to generate physiological information used to generate outputs that reflect a pattern of the user's heartbeat. For example, the self-contained handheld device also includes a vibration motor configured to cause the self-contained handheld device to vibrate in accordance with the physiological information, and a light source configured to illuminate at least a portion of the self-contained handheld device in accordance with the physiological information.

Embodiments also include a method performed by a standalone computing device. The method includes detecting signals indicative of a user's physiological activity obtained from the user while the user is holding the standalone computing device in the palm of the user's hand, generating physiological information based on the obtained signals indicative of the physiological activity. The physiological information includes an indication of a pattern of the user's bodily function occurring while the standalone computing device is held in the palm of the user's hand. The method further includes generating a physically perceptible output based on the physiological information such that the physically perceptible output is a physical rendering of the pattern of the user's bodily function.

Embodiments also include a method of pairing a computing device with a handheld wellness device. The method includes causing display, via a display of the computing device, of a visual prompt defining a region of the display of the computing device, detecting that the handheld wellness device is positioned relative to the defined region of the display of the visual prompt, causing the computing device to render an output that is perceptible by a sensor of the handheld wellness device, monitoring for an indication of the rendered output from the handheld wellness device, and establishing a wireless communications link between the computing device and the handheld wellness device in response to detecting the indication of the rendered output.

In some embodiments, wherein detecting that the handheld wellness device is positioned relative to the defined region of the display of the visual prompt includes detecting that the handheld wellness device is positioned within a threshold proximity to the defined region of the display of the visual prompt.

In some embodiments, causing the computing device to render the output that is perceptible by the sensor of the handheld wellness device includes causing the computing device to vibrate at a predetermined pattern that is perceptible by the sensor of the handheld wellness device.

In some embodiments, monitoring for the indication of the rendered output from the handheld wellness device includes monitoring one or more advertising channels of a wireless communication signal for the indication of the rendered output.

Embodiments include a method performed by a handheld wellness device for pairing a computing device with the handheld wellness device. The method includes detecting one or more computing devices via one or more wireless communications channels, broadcasting a signal via the one or more wireless communications channels to the one or more computing devices, detecting an output from a particular computing device of the one or more computing devices. The output can include an indication of the transmitted signal. The method further includes establishing a wireless communications link between the particular computing device and the handheld wellness device in response to detecting the output.

In some embodiments, detecting one or more computing devices includes detecting multiple computing devices.

In some embodiments, the particular computing device is a smartphone.

In some embodiments, the broadcasted signal includes a predetermined code used to establish the wireless communications link.

In some embodiments, the method further includes monitoring physiological activity of a user while the user is holding the handheld wellness device in the user's hand. In some embodiments, the monitored physiological activity is indicative of a breathing pattern of the user obtained in real-time while the user is holding the handheld wellness device in the user's hand. In some embodiments, the monitored physiological activity is indicative of a heart rate of the user obtained in real-time while the user is holding the handheld wellness device in the user's hand. In some embodiments, the method further includes transmitting information indicative of the monitored physiological activity to the particular computing device and causing the particular computing device to store the transmitted information indicative of the monitored physiological activity.

In some embodiments, the method further includes generating multisensory physically perceptible output while a user is holding the handheld wellness device in the user's hand. The multisensory physically perceptible output is based on the monitored physiological activity. In some embodiments, the multisensory physically perceptible output includes a vibration pattern generated in real-time based on the monitored physiological activity. In some embodiments, the multisensory physically perceptible output includes an illumination pattern that dynamically changes in real-time to guide a breathing rate of the user. In some embodiments, the multisensory physically perceptible output includes an illumination pattern generated in real-time based on the monitored physiological activity. In some embodiments, the multisensory physically perceptible output includes a vibration pattern generated in real-time based on the monitored physiological activity and an illumination pattern synchronized with the vibration pattern. In some embodiments, the multisensory physically perceptible output is configurable at the particular computing device.

In some embodiments, the multisensory physically perceptible output is selected at the particular computing device and communicated to the handheld wellness device. In some embodiments, the selected multisensory physically perceptible output is one of multiple modes selected at the particular computing device. In some embodiments, the selected multisensory physically perceptible output is an indication of a person's physiological activity stored in a memory of the particular computing device. In some embodiments, the selected multisensory physically perceptible output is shared by a remote handheld wellness device in real-time as selected at the particular computing device.

In some embodiments, the handheld wellness device is a local wellness device, and the method further includes receiving, by the local wellness device via the particular computing device, an indication of a monitored physiological activity of a remote user holding a remote handheld wellness device and generating multisensory physically perceptible output while a local user is holding the local wellness device. The multisensory physically perceptible output is based on the monitored physiological activity of the remote user.

In some embodiments, the method further includes monitoring physiological activity of a user while the user is holding the handheld wellness device in the user's hand and generating multisensory physically perceptible output simultaneously while monitoring the physiological activity of the user while the user is holding the handheld wellness device. The multisensory physically perceptible output is based on the monitored physiological activity.

In some embodiments, the method further includes receiving, via the particular computing device, data indicative of a physiological activity of a person, wherein the handheld wellness device is configured to generate multisensory physically perceptible output based on the data indicative of the physiological activity of the person while the user is holding the handheld wellness device.

In some embodiments, the method further includes monitoring physiological activity of a user while the user is holding the handheld wellness device, transmitting, via the particular computing device, data indicative of the monitored physiological activity to a cloud-based storage such that the data indicative of the monitored physiological activity is retrievable from the cloud-based storage, and receiving, via the particular computing device, data indicative of a physiological activity stored in the cloud-based storage, wherein the handheld wellness device is configured to generate multisensory physically perceptible output based on the received data indicative of the physiological activity.

In some embodiments, the received data that is indicative of the physiological activity is the data indicative of the monitored physiological activity stored in the cloud-based storage. In some embodiments, the received data that is indicative of the physiological activity is physiological activity of another user as obtained with the remote handheld wellness device and stored to the cloud-based storage. In some embodiments, a copy of the received data indicative of the physiological activity is publicly available to any user accessing the cloud-based storage. In some embodiments, a copy of the received data indicative of the physiological activity is only available from the cloud-based storage by an authorized user.

This Summary is provided to introduce a selection of concepts in a simplified form that is further described below in the Detailed Description. This Summary is not intended to identify key features or essential features of the disclosed subject matter, nor is it intended to be used to limit the scope of the disclosed subject matter. Other aspects of the disclosed subject matter will be apparent from the accompanying Figures and Detailed Description.

### BRIEF DESCRIPTION OF THE DRAWINGS

FIG. 1 illustrates a standalone handheld wellness device operable to monitor physiological activity of a user and generate physically perceptible output based on the monitored physiological activity according to some embodiments of the present disclosure;

FIG. 2A illustrates an exploded view of the wellness device according to some embodiments of the present disclosure;

FIG. 2B illustrates another exploded view of the wellness device according to some embodiments of the present disclosure;

FIG. 3A illustrates a wellness device embodied as a handheld sphere according to some embodiments of the present disclosure;

FIG. 3B illustrates a wellness device embodied in a component of a chair according to some embodiments of the present disclosure;

FIG. 3C illustrates a wellness device embodied as a stuffed animal according to some embodiments of the present disclosure;

FIG. 3D illustrates a wellness device embodied as a necklace according to some embodiments of the present disclosure;

FIG. 4A illustrates a screenshot of a login screen of the mobile app rendered on a mobile device according to some embodiments of the present disclosure;

FIG. 4B illustrates a screenshot of a signup screen of the mobile app according to some embodiments of the present disclosure;

FIG. 4C illustrates a screenshot of a settings screen of the mobile app according to some embodiments of the present disclosure;

FIG. 4D illustrates a screenshot of a “buy” screen of the mobile app that enables a user to purchase a mode of operation profile according to some embodiments of the present disclosure;

FIG. 4E illustrates a screenshot of a “self” screen of the mobile app that enables a user to manage the user's physiological information according to some embodiments of the present disclosure;

FIG. 4F illustrates a screenshot of a “shared” screen of the mobile app that enables a user to access shared physiological activity data according to some embodiments of the present disclosure;

FIG. 4G illustrates a screenshot of a “saved” screen of the mobile app of saved physiological activity data according to some embodiments of the present disclosure;

FIG. 4H illustrates a screenshot of a “detailed save” screen of the mobile app that enables a user to access physiological activity data associated with events for a particular person according to some embodiments of the present disclosure;

FIG. 4I illustrates a screenshot of an “add heartbeat” screen of the mobile app for saving a user's heartbeat according to some embodiments of the present disclosure;

FIG. 4J illustrates a screenshot of a “detailed add heartbeat” screen of the mobile app for saving a heartbeat according to some embodiments of the present disclosure;

FIG. 4K illustrates a screenshot of a “shared” screen of the mobile app for sharing a saved or real-time heartbeat with other people;

FIG. 4L illustrates a “shared” screen of the mobile app for selecting heartbeats shared by other people;

FIG. 5 is a diagram illustrating wireless bonding between the computing device and the wellness device according to some embodiments of the present disclosure;

FIG. 6A illustrates a screenshot of a wireless bonding screen of the mobile app before pairing with a wellness device;

FIG. 6B illustrates a screenshot of a wireless bonding screen of the mobile app while pairing with the wellness device;

FIG. 6C illustrates a screenshot of a wireless bonding screen of the mobile app prompting a user to accept or deny pairing with the wellness device;

FIG. 6D illustrates a screenshot of a wireless bonding screen of the mobile app after pairing with the wellness device is completed;

FIG. 7 is a block diagram illustrating components of the wellness device according to some embodiments of the present disclosure; and

FIG. 8 is a block diagram illustrating components of a computing device in which embodiments of the present disclosure can be implemented.

### DETAILED DESCRIPTION

The embodiments set forth below represent the necessary information to enable those skilled in the art to practice the embodiments and illustrate the best mode of practicing the embodiments. Upon reading the following description, in light of the accompanying Figures, those skilled in the art will understand the concepts of the disclosure and will recognize applications of these concepts that are not particularly addressed herein. It should be understood that these concepts and applications fall within the scope of the disclosure and the accompanying embodiments.

The purpose of the terminology used herein is only for describing embodiments and is not intended to limit the scope of the disclosure. Where context permits, words using the singular or plural form may also include the plural or singular form, respectively.

As used herein, unless specifically stated otherwise, terms such as “processing,” “computing,” “calculating,” “determining,” “displaying,” “generating” or the like, refer to actions and processes of an electronic computing device that manipulates and transforms data, represented as physical (electronic) quantities within the computer's memory or registers, into other data similarly represented as physical quantities within the computer's memory, registers, or other such storage medium, transmission or display devices.

As used herein, the terms “connected,” “coupled,” or variants thereof refer to any connection or coupling, either direct or indirect, between two or more elements. The coupling or connection between the elements can be physical, logical or a combination thereof.

The disclosed embodiments include a standalone handheld wellness computing device (“wellness device”). As used herein, the term “standalone” may refer to a device that can provide a desired function without ever needing to communicatively couple to any network or another device. In other words, a standalone device can operate independent of any other device to generate an output based on an input without communicating data to any network or another device. As used herein, the term “wellness” may refer to a device having a function designed to improve the physical and/or emotional state of a user. For example, a wellness device can process inputs/outputs (I/O) meant to reduce a user's level of loneliness, tension, stress, or anxiety. As used herein, the term “handheld” may refer to the size of the device relative to the user's hand. In particular, a handheld device is relatively small such that a person could hold the handheld device with one hand. Accordingly, the handheld device is typically portable and can be carried by a person.

In an embodiment, the wellness device can provide biofeedback by monitoring a normally automatic bodily function and rendering outputs to train a person to acquire voluntary control of that function. The I/O processing can all be contained in the wellness device. For example, the wellness device can generate multisensory outputs based entirely on inputs detected exclusively by the wellness device, independent of any other network or computing device. In contrast, existing biofeedback systems have multiple distributed components including sensors electrically coupled to monitoring devices and display outputs. Moreover, the wellness device is a standalone consumer device that does not require a skilled person to setup or operate.

Unlike existing consumer activity trackers, the disclosed wellness device does not need to be worn by a user or paired with another device to setup or operate as intended. Instead, the wellness device could be held for a period of time and generate physically perceptible output that can be experienced by a user without holding or wearing the device. For example, the disclosed wellness device could detect a heartbeat pattern of a user holding the wellness device, and can render an illumination pattern at a different time after the user has left the wellness device atop a table.

The wellness device could be designed to appear like a natural object such as a river stone that is sufficiently small enough to comfortably fit in the palm of a user's hand. As such, the wellness device would not appear like a gadget such as a wristwatch, smartphone, or another mobile electronic device. Instead, the appearance of the wellness device could promote wellness by resembling an object designed to induce a connection with natural objects found in nature. However, the wellness device is not limited to resembling natural objects. Instead, it could appear like a technological gadget or any other object.

The wellness device includes one or more mechanisms to receive inputs of a user. For example, the wellness device can include one or more sensors that detect a user's physiological activity as inputs received while the user holds the wellness device. The wellness device also includes one or more mechanisms to render physically perceptible outputs. For example, the wellness device can include one or more actuators that generate haptic outputs or light sources that generate illumination patterns.

The received inputs can be processed to produce physiological information used to generate corresponding outputs. For example, the rhythm of a user's heartbeats could be detected and recorded as physiological information that is used to generate physically perceptible outputs such as vibrations and/or illumination or color patterns rendered in accordance with the rhythm of the user's heartbeats. As such, the wellness device could produce multisensory outputs synchronized in accordance with physiological information produced from inputs. In some embodiments, the wellness device can render the outputs simultaneous or near simultaneous in time with the received inputs, in response to the received inputs. As such, a user could experience touching, seeing, and/or hearing a physiological activity that is otherwise imperceptible to the user.

The inputs are indicative of a person's physiological behavior or activity, which may refer to a bodily function that is not readily perceptible by a person. Although inputs can be obtained by sensors tracking the physiological activity of a user holding the wellness device, the disclosed embodiments are not so limited. For example, the inputs could be processed by a first wellness device to generate physiological information, and communicated over a network to a second wellness device, which can render the physiological information shared by the first wellness device.

Examples of physiological information can include a rhythm or pattern of a person's heart (e.g., heart rate) measured with a sensor of the wellness device in direct contact (e.g., skin contact) or indirect contact (e.g., through a glove) with a person's hand. In some embodiments, the physiological information represents bodily functions controlled by the autonomic nervous system, which is a control system that acts largely unconsciously and regulates bodily functions such as the heart rate, digestion, respiratory rate, pupillary response, urination, and sexual arousal. In particular, this system is the primary mechanism in control of the fight-or-flight response.

The outputs are rendered based on the physiological information to produce outputs in a physically format that can be perceived by a user of the wellness device. Examples of physically perceptible outputs include visual, tactile, and/or audible outputs that can be experienced by a user of the wellness device. For example, physically perceptible outputs can include a vibration pattern having the same or similar pattern or rhythm (e.g., frequency) of the monitored user's heartbeat. In some embodiments, the outputs may include one or more light sources that illuminate at least a portion of the wellness device in accordance with an illumination pattern that is synchronized with the heartbeat rendered by the wellness device. As such, the user holding the wellness device could see and feel outputs tracking the user's heartbeat. In some embodiments, the illumination pattern could reflect a respiratory rate measured of the user holding the wellness device or reflect a program meant to guide the user's breathing pattern.

For example, the illumination pattern can be used to guide a user's breathing to a specific scientifically validated rate. Examples of breathing rates include box breathing: a 4 second inhale (lights get brighter), 4 second pause (lights stay bright), 4 second exhale (lights get dimmer), 4 second pause (lights stay dim). The user (or a remote user such as a therapist) can select between a plurality of breathing rates to find the one that is most helpful to the user. Other example breathing rates include a 4 second inhale, 7 second pause, and 8 second exhale.

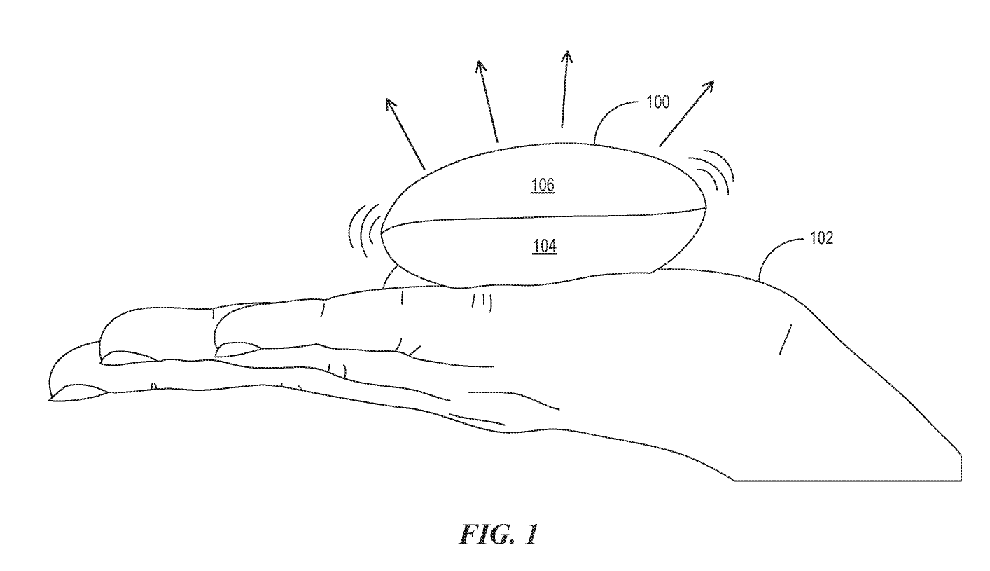

FIG. 1 illustrates an embodiment of a wellness device operable to monitor physiological activity of a user and generate physically perceptible output based on monitored physiological activity. As shown, the wellness device 100 is sufficiently small enough to be held on the palm of a user's hand 102. The wellness device 100 is shaped as a relatively smooth (e.g., edgeless), small stone. More particularly, the design of the wellness device 100 is such that it appears like a natural object such as a river stone rather than an electronic gadget. The size and shape of the illustrated embodiment is such that the wellness device 100 can be concealed by gripping the device, or can fit into the pocket of an article of clothing.

As shown, the wellness device 100 is formed of two main exterior portions joined to form a contiguous exterior body. A lower portion 104 can include one or more sensors configured to detect inputs. For example, the wellness device 100 can include a sensor partly contained in the housing of the wellness device 100 and partly protruding along the surface of the lower portion 104. The sensor may be in contact with the user's hand 102 to detect physiological activity of the user such as a heartbeat rate. The sensors enable the wellness device 100 to collect physiological data via the user's hand 102. The wellness device 100 can also include a memory to store the collected physiological data or physiological information obtained by processing the physiological data.

The wellness device 100 can include other sensors positioned anywhere within or on the surface of the wellness device 100 to detect, for example, sounds, movement, or capture images used to generate physiological information. For example, the wellness device 100 can have a sensor that is completely contained within the housing without any portion protruding out on the lower portion 104. This fully contained sensor can sense physiological activity without needing to be in direct contact with the user's skin. For example, the wellness device 100 can include an accelerometer used to detect breathing or tension (vibration) while being held by the user, without contacting the user's skin. Hence, the wellness device 100 can include a combination of sensors that do and/or do not require skin contact, are LED or galvanic sensors that partially protrude from the wellness device 100, sensors that only sense movement while being held by the user, etc.

The wellness device 100 can use the physiological information to generate outputs that are readily perceived by the user. The outputs may be generated in real-time, near real-time, or later in time relative to collecting inputs or generating physiological information. As used herein, a “real-time” process may refer to a process that includes a negligible delay that is customarily expected in typical real-time processes. In one example, in a real-time biofeedback mode, the wellness device 100 can generate outputs based on inputs being received by the wellness device 100 in real-time or near real-time. In another biofeedback mode, the wellness device 100 can generate outputs based on inputs that were previously recorded for subsequent rendering by the wellness device 100. As such, the wellness device 100 can be used to train the heartbeat of a user holding the wellness device 100. For example, the wellness device can initially generate multisensory outputs having a pattern that matches the user's current heartbeat pattern and then changes the frequency or magnitude of the pattern of outputs to induce an increase or decrease in the frequency or magnitude of heartbeat or breathing patterns of the user holding the wellness device 100.

In a share mode, the wellness device 100 can generate outputs for a user based on physiological information obtained of another user. For example, a first user could use a first wellness device to obtain physiological information of the first user. The first user can be any person or animal such as a user's pet. The physiological information can be used to generate outputs perceived by a second user of the same first wellness device or of a second wellness device. For example, physiological information could be communicated over a network to another wellness device used by another user. The physiological information could be shared in real-time or near real-time or could be shared later in time after being recorded by a wellness device.

The wellness device 100 may be enabled to render multisensory outputs including visual, audible, and/or tactile outputs. The wellness device 100 could include actuators to produce haptic feedback based on physiological information. For example, the wellness device 100 can vibrate with a pattern that tracks a user's heartbeat. In other example, the wellness device 100 could produce audio feedback based on physiological information such as playing a song or other audio with a beat that tracks a rhythm of the user's heartbeat. In yet another example, the wellness device 100 could emit light with an illumination pattern that tracks the user's heartbeat.

For example, the upper portion 106 of the wellness device 100 includes a panel or surface that is transparent to visible light emitted from one or more internal light sources. The emitted light can be rendered from the upper portion 106 in the field of view of the user holding the wellness device 100 and with an illumination pattern that tracks physiological information such as a heartbeat rate and/or intensity. For example, the wellness device 100 can emit light that switches on/off at the same rate as a user's heartbeat and/or changes colors depending on the rate or intensity of the user's heartbeat. For example, the color of the emitted light could depend on whether a heartbeat rate is within a predetermined range of values.

In a biofeedback mode, visual feedback or any combination of multisensory outputs could be rendered to help guide a user to breathe in a desired manner by adapting outputs to detected physiological activity, and then gradually change the outputs to induce the user to relax. The colors emitted by the upper portion 106 may be selected based on whether the detected heartbeat rate is within a predetermined range. For example, the wellness device 100 may generate an illumination pattern that tracks a detected heartbeat rate and at a particular color for a heartbeat within a range of rates. For example, the wellness device 100 may emit blue light to indicate a “relaxed” state when a relatively low heartbeat rate is detected, and emit a yellow light to indicate an “elevated” state when a relatively higher heartbeat rate is detected.

The lower portion 104 and the upper portion 106 of the wellness device 100 could be made of plastic, metal, glass, any other materials, or any combination of materials and colors. For example, at least a portion of the wellness device 100 could be made of any combination of silicone, hemp, glass (including stained glass), wood (e.g., cherry, maple, walnut, hickory, rosewood, macacauba, pine), stone, crystal, gold, stainless steel, aluminum, ceramic, fabrics, or any other material that gives the wellness device a natural or elegant appearance rather than appearing like an awkward gadget. More broadly, a wellness device can be designed to appear natural or mimic something in nature, to be non-threatening, non-technology, sleek and elegant rather than awkward, etc. A wellness device can be made of material that fosters a sense of natural connection such as fabric, gold, wood, etc. Hence, a wellness device can be designed with whatever connects a user with nature rather than connecting the user to friends on a TWITTER feed or FACEBOOK. Thus, one or more portions made of one or more materials that collectively form the wellness device 100 held in the user's hand 102. The wellness device 100 is in contact with the user's hand 102 so that any number of sensors of the same or different types can monitor and track the user while the user is holding the wellness device 100.

As such, the wellness device 100 can receive inputs from a sensor of the lower portion 104 or based on a computation operation performed by the wellness device 100, and render some outputs from the upper portion 106 of the wellness device 100. The inputs can include signals generated by one or more sensors located on the lower portion 104 or any other portion of the wellness device 100. The signals can be processed to generate measurements as physiological information indicative of the user's physiological activity. Examples of physiological activity include heartbeat rate, heart rate variability, muscle tone, breathing rate, skin conductance, and the like. The outputs rendered by the wellness device 100 can be generated based on the measurements of the physiological activity. As such, the wellness device 100 can provide real-time or near real-time biofeedback to the user. The feedback allows the user to gain greater awareness of many physiological functions, and can train the user to change the physiological functions.

The wellness device 100 can include any number of input mechanisms such as the aforementioned one or more sensors. An input mechanism can include electronic components that detect physiological or environmental conditions or events. Hence, the wellness device 100 can operate as a biometric monitoring device that can be used to, for example, monitor a user's heartbeat rate, heart rate variability, breathing rate, muscle tone, skin conductance, and the like. The particular input mechanisms described herein are non-limiting examples meant to illustrate a number of possible mechanisms that can be included in the wellness device 100. The wellness device 100 can include a variety of other types of input mechanisms that are known to persons skilled in the art, but not described herein for the sake of brevity.

The wellness device 100 can include any type, number, or combination of input mechanisms to detect physiological activity as received inputs from a user or the user's environment. The input mechanisms may be active input mechanisms and/or passive mechanisms. An active input mechanism receives deliberate and voluntary inputs from a user seeking to effect a particular action or output such as tapping or shaking the wellness device 100 to activate its recording capabilities or to navigate through available modes of operation. An example of an active mechanism includes a touch sensitive surface on the wellness device 100 that can detect touch-based gestures such as a swipe or tap. Another example includes gesture inputs detected by moving or shaking the wellness device 100 in space. For example, an accelerometer of the wellness device 100 can detect when the wellness device 100 has been shaken or tapped to effect a desired action. Yet another example of an input mechanism includes a microphone of the wellness device 100 configured to detect sounds or voice commands, and produce actions or outputs accordingly. Hence, the wellness device 100 does not require being actively held in a user's hand. Instead, for example, the wellness device 100 could be in contact with any part of a user's body such as the user's chest, forehead, leg, or another body part.

A passive input mechanism receives passive inputs from the user including signals indicative of physiological activity that are processed to produce physiological information. For example, a passive input mechanism may include a heartbeat sensor located at the lower portion 104 of the wellness device 100 that can detect the user's heartbeat pattern (e.g., heartbeat rate) when the user is holding the wellness device 100. Other examples of passive input mechanisms include sensors configured to determine muscle tone by detecting muscle action potentials, a thermistor configured to detect the user's skin temperature, and a blood sensor that emits infrared (IR) light onto the user's skin and detects blood-flow or blood-oxygen levels based on the amount of IR light reflected back. In yet another example, the wellness device 100 can include a galvanometer that measures skin electrical activity directly (i.e., conductance and the potential) and indirectly (i.e., skin resistance) to assess the user's stress levels. Other examples of input mechanisms include a proximity sensor configured to determine whether a user is holding the wellness device 100 or, for example, whether the device is placed somewhere in a room proximate to the user. Another example of an input mechanism is an altimeter that can be used to measure the rise and fall of the user's hand, which can be used to determine a breathing pattern.

The wellness device 100 includes mechanisms to process received inputs and generate outputs based on the processed inputs. For example, the wellness device 100 may include program instructions stored in a memory and executed by a processor to generate physiological information based on inputs indicative of a user's physiological activity. The physiological information may be produced from raw data collected by sensors that has been processed to identify certain useful physiological information. For example, the wellness device 100 could deconstruct received inputs into component signals that are compared against known indicators to identify those components signals that are indicative of certain physiological activity such as heartbeats. The received inputs could undergo a transformation and filtering process to improve the quality of the desired signal components and perform correction techniques to correct or supplement the received inputs in order to produce usable physiological information for generating physically perceptible outputs that track the received inputs.

Embodiments of the wellness device 100 could have a reduced number of components that enable receiving inputs and rendering outputs but offload producing physiological information to one or more other computing devices. For example, the wellness device 100 could be communicatively coupled to a smartphone that remotely processes physiological data collected by the wellness device 100 as received inputs. The smartphone could produce physiological information that is transmitted to the wellness device 100 with instructions for generating outputs based on the physiological information.

In some embodiments, the wellness device 100 could have a fewer number of components than enable rendering outputs but offload the functions of receiving inputs and processing the received inputs to produce physiological information. For example, the wellness device 100 could be communicatively coupled with an activity tracker worn by a user that detects and transmits physiological data or physiological information to the wellness device 100. In some embodiments, the activity tracker can transmit the physiological data to another computing device (e.g., smartphone) that processes the data to produce physiological information that is then transmitted to the wellness device 100 with instructions for generating outputs based on the physiological information. Other embodiments, can offload any combination of function to one or more other devices that can be communicatively coupled to wellness device 100. A wellness device with fewer components could have a reduced cost that could expand the appeal of the wellness device to a broader audience seeking a lower price point.

The output mechanisms can generate any number of physically perceptible outputs that indicate information other than physiological information. For example, the wellness device 100 can include light sources operable to emit light of different intensities and colors that can turn on and off in accordance with a specified pattern to indicate a status of the wellness device 100 such as a low battery status or charging status. Examples of output mechanisms other than a vibration motor configured to cause the wellness device to vibrate or a light source includes a heating or cooling element configured to change the temperature of the wellness device 100, and a speaker configured to generate audio. The wellness device 100 can include these and other output mechanisms known to persons skilled in the art but not described herein for the sake of brevity.

As indicated above, the outputs of the wellness device 100 may be based on the inputs received from the user in real-time or near real-time. For example, outputs may be indicative of a heartbeat pattern and breathing cycles, which can be derived from the heartbeat pattern (e.g., the heart rate changes as a person inhales and exhales). In some embodiments, the outputs can be indicative of blended heartbeats, such as the heartbeat of a pregnant woman and the baby in her womb, or blended heartbeats of two users. In other embodiments, the outputs can be indicative of blended breathing cycles, such as the breathing of lovers in a long-distance relationship. In some embodiments, the wellness device 100 can separate blended heartbeats into components to, for example, render the heartbeat pattern of a baby in the womb by placing the wellness device 100 over the user's womb or even via the user's hand.

In some embodiments, the outputs could be influenced by environmental factors. For example, the wellness device 100 can include sensors that detect environmental data. The environmental data can affect outputs of the wellness device 100 by, for example, changing the illumination patterns and vibration patterns to synchronize or track to inputs from the user's environment such as music, lights, humidity, temperature, and noise levels. For example, the wellness device 100 can include an ambient light sensor, and adjust the light emitted by the wellness device based on the detected ambient light. For example, the intensity of the light emitted by the wellness device 100 could be decreased in darker environments and increased in relatively brighter environments.

In some embodiments, the outputs can be based on information stored in a memory of the wellness device 100 or stored in a remote cloud-based memory that is communicatively coupled to the wellness device 100 over a network. A user can cause the wellness device 100 to record heartbeat data in a memory device, and the same user or another user can cause the wellness device 100 to generate physically perceptible outputs that reflect the recorded heartbeat data. As such, a user can record physiological activity and then render outputs indicative of the physiological activity at a later time. For example, family members or loved ones can record and share heartbeat data that is rendered by wellness devices.

As such, the wellness device 100 can provide a sense of social connectedness among people by “sharing” heartbeat data and saving the physiological representations of precious moments in their relationships. In some embodiments, the outputs can be based on information communicated from a remote source over a network. For example, the wellness device 100 may include a wireless transceiver or port that can receive a cable to connect with another wellness device or a computing device such as a smartphone or personal computer. The wellness device 100 can be used to communicate data about physiological activity in real-time, or data stored in memory, to another device. For example, information indicative of a user's heart rate and breathing cycles can be transmitted to another electronic device over a wireless link. In another example, information indicative of another user's heartbeat rate and/or breathing cycles can be received from an electronic device over the wireless link and rendered by the wellness device 100. As such, a user of the wellness device 100 can experience shared physically perceptible outputs of another user's physiological activity.

In some embodiments, the wellness device 100 can broadcast physiological information to multiple other wellness devices over wireless links to enable all the wireless devices to render the same outputs simultaneously. In some embodiments, the physiological information can be collected by the wellness device 100, communicated over a network to another wellness device that renders the collected data. As such, for example, the wellness device 100 can be part of a baby monitoring system that allows a user to monitor the heartbeat data of a baby having the wellness device in contact with the baby. In some embodiments, the wellness device 100 can be programmed to render outputs as part of a guided tour of a museum or an art installation, or the outputs can be synchronized to music, or provide a meditation timer.

In some embodiments, multiple wellness devices may simultaneously share physiological information of detected physiological activity (e.g., heartbeats) with each other as the physiological activity is detected. For example, in an embodiment, heartbeat data of a first user detected by a first wellness device may be shared in real time with a second wellness device which is then experienced as an output by a second user of the second wellness device. Simultaneously, heartbeat data of the second user detected by the second wellness device may be shared in real-time with the first wellness device which is then experienced as an output by the first user of the first wellness device. In this way, two or more users, each with a wellness device, may simultaneously experience the heartbeats of each other. As an illustrative example, using two wellness devices, a couple may simultaneously experience outputs that reflect each other's heartbeats, for example, during a therapy session.

The simultaneous sharing of heartbeat data or other physiological activity between multiple wellness devices may also allow for the gathering of information on how experiencing one person's heartbeat affects another person's heartbeat. Consider again the above described scenario of two wellness devices simultaneously sharing the respective heartbeats of the two users. In some embodiments, the simultaneously shared heartbeats may be tracked and analyzed, for example, to discover correlations between the physiological activity of one person in response to experiencing a rendered output based on the physiological output of another. This tracking and analysis may be performed by one or more of the two wellness devices and/or anther computing device communicatively coupled to one or more of the two wellness devices.

In some embodiments, the physiological activity of multiple users detected by multiple wellness devices may be aggregated and rendered by other wellness devices as a single output, for example, representing an average or collective physiological activity. For example, consider a scenario in which one person is giving a presentation to a crowd of multiple other people and that each person in this scenario has a wellness device. As previously described, in some embodiments, the heartbeat of the presenter may be detected by a first wellness device and broadcast to the multiple wellness devices held by the people in the audience. Simultaneously, the heartbeats of the multiple people in the audience may be detected by their respective wellness devices and aggregated to render an output representing an average or collective heartbeat of the audience which can then be shared, for example with the wellness device of the presenter. In some embodiments the aggregation of the multiple heartbeats and rendering of an output based on the aggregated heartbeats may be performed by the wellness device of the presenter, any of the wellness devices of the multiple audience members, another computing device communicatively coupled to the multiple wellness devices of the audience, and/or by the collective computing resources of the multiple wellness devices in this scenario, for example using distributed computing techniques.

In some embodiments, the wellness device 100 can transmit or broadcast active user inputs to other computing devices or wellness devices, for example as a means for communication between users. For example, in an embodiment a wellness device include a capacitive or resistive touch surface through which a user can provide touch input. The touch surface may further include backlighting (e.g., through the use of light emitting diodes (LEDs)) that provide an output via the surface in response to the users touch. As the user touches the surface of the wellness device, lights beneath the surface may activate at positions corresponding to the user's touch. In this manner, the user may draw symbols or patterns via the surface that can then be transmitted to other computing devices or wellness devices. For example, a user may draw a symbol of a heart on the touch surface of a first wellness device and initiate transmissions of the heart symbol by, for example, shaking or gesturing with the first wellness device. In response, the first wellness device may transmit the heart symbol over a computer network to another computing device or wellness device that renders the drawn heart symbol on the receiving wellness device.

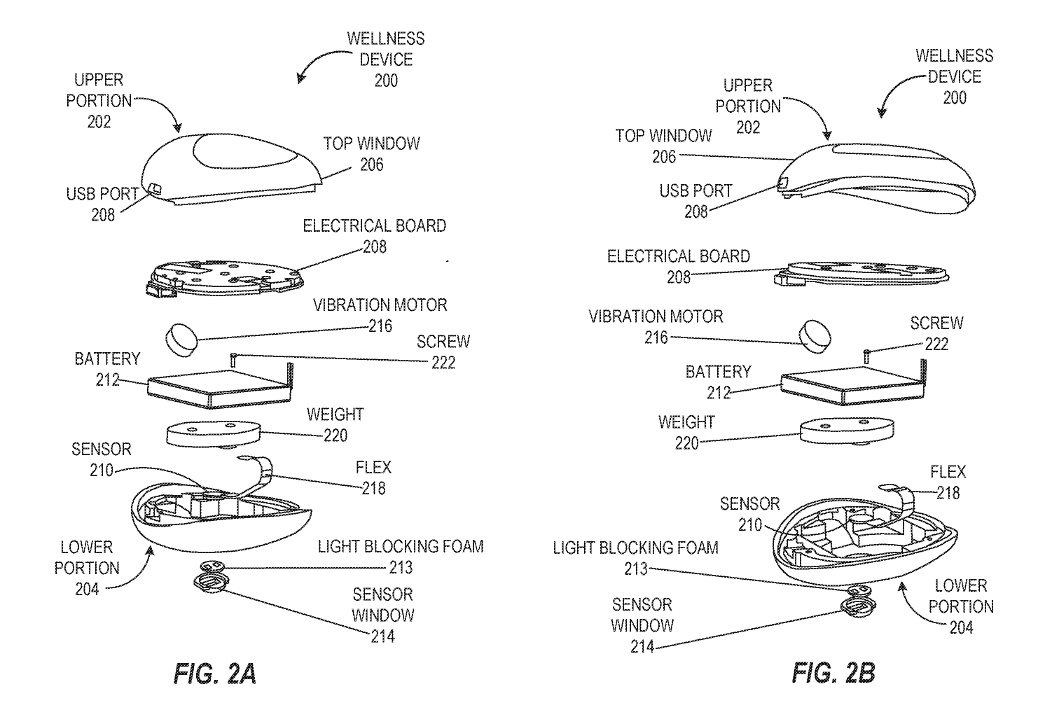

FIGS. 2A and 2B illustrate different perspectives of an exploded view of a wellness device 200 according to some embodiments of the present disclosure. The wellness device 200 includes several electronic and mechanical components in a body formed by joining an upper portion 202 with a lower portion 204 of the wellness device 200. In other words, the upper portion 202 and lower portion 204 collectively form an exterior shell that can include one or more input mechanisms, output mechanisms, and processing circuitry for producing physiological information used for rendering output based on inputs of physiological activity.

The upper portion 202 includes a window 206 that could cover an area coextensive with or less than the exterior surface of the upper portion 202. The window 206 is at least semi-transparent to visible light emitted by light sources on the electronic circuit board 208. For example, the electronic circuit board 208 may include LEDs that can emit light through the window 206 in the field of view of a user holding the wellness device 200.

The upper portion 202 may also include an opening for a universal serial bus (USB) port 208 which can be used to receive a USB cable that can connect the wellness device 200 to another device. In other embodiments, the wellness device does not have a USB port 208 and can communicate with another device wirelessly. A wired or wireless connection can connect the wellness device 200 to a power source that charges a battery 212.

The lower portion 204 has an opening that receives a sensor 210 configured to detect physiological activity of a user holding the wellness device 200. The sensor 210 is secured flush to the surface of the lower portion 204 with a light blocking foam 213 configured to go around the sensor 210. A sensor window 214 secures the light blocking foam 213 to the sensor 210 at the bottom of the lower portion 204 such that the sensor 210 is in contact or proximate to a user's hand holding the wellness device 200.

The lower portion 204 fits together with the upper portion 202 to form an enclosure that houses various mechanical and electronic components of the wellness device 200. In some embodiments, the upper portion 202 and the lower portion 204 snap securely to lock together. In some embodiments, the upper portion 202 and the lower portion 204 can be secured together with one or more screws or an adhesive. In some embodiments the upper portion 202 and the lower portion 204 of the wellness device 200 may include areas that function as a capacitive and/or resistive touch surface to receive touch inputs used to control functions of the wellness device 200.

The wellness device 200 houses the electronic circuit board 208 that includes various electronics such as a microcontroller, battery power regulating chips, memory chip, ambient light sensor, LEDs, a Bluetooth module, and the like. The electronic circuit board 208 is coupled to a vibration motor 216 configured to cause the wellness device 200 to vibrate as a physically perceptible output. Examples of a vibration motor include an eccentric rotating mass (ERM) or linear resonant actuator (LRA) type vibration motor.

The battery 212 housed in the wellness device 200 is coupled to the electronic circuit board 208 and the vibration motor 216. Examples of the battery 212 include a lithium ion battery or any other type of rechargeable battery. In some embodiments, the battery 212 can be recharged via the USB port 208. The wellness device 200 includes a flex structure 218 that includes the sensor 210 (e.g., a photoplethysmography (PPG) heartbeat sensor) facing the opening at the lower portion 204 of the wellness device 200 and on which the electronic circuit board 208 is mounted. As such, for example, a heartbeat monitoring sensor is coupled to the components of the electronic circuit board 208. The wellness device 200 includes a weight 220 to give the wellness device a realistic weight of a natural rock of similar size. Lastly, the wellness device 200 can include one more screws such as screw 222 to couple components to each other or to a frame or body of the wellness device 200.

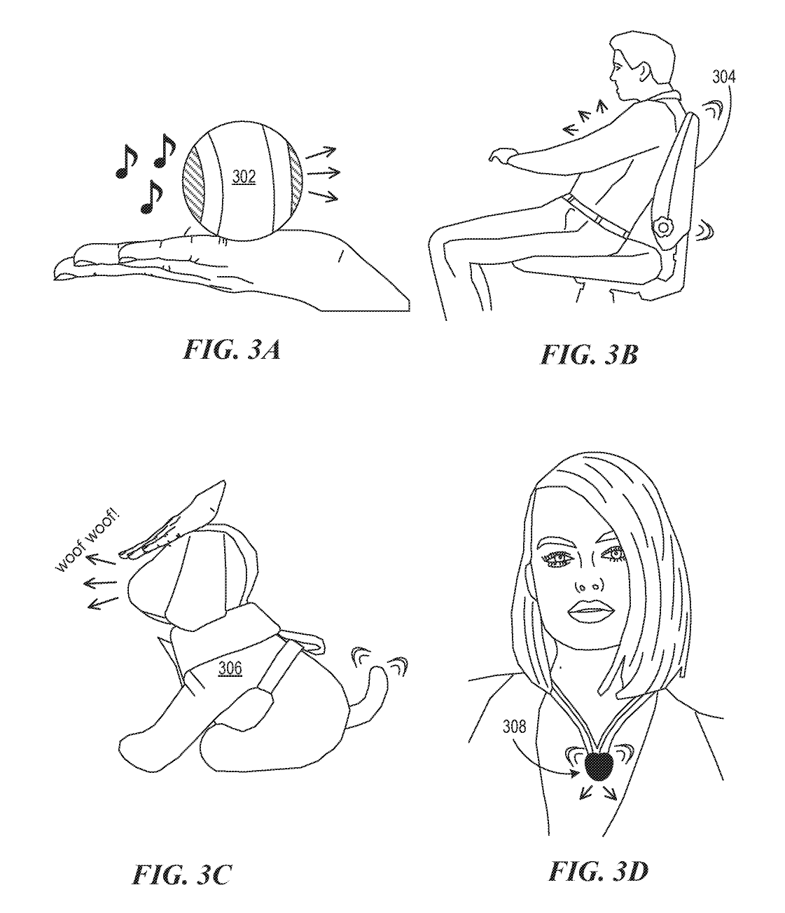

Although FIGS. 1 and 2 illustrate a wellness device embodied as a handheld device with a natural river stone appearance, the disclosure is not limited to this particular physical design. For example, FIGS. 3A through 3D illustrate embodiments of a wellness device with other physical designs.

FIG. 3A illustrates a wellness device embodied as a handheld sphere 302 that has multisensory outputs including light and music that is rendered in accordance with detected physical activity of a user holding the handheld sphere 302. For example, the handheld sphere 302 may include sensors that detect physiological activity via a user's hand or movement by the user. The handheld sphere 302 could play a particular type of music based on, for example, whether the user is participating an aerobic exercise or weight training.

FIG. 3B illustrates a wellness device embodied in a component of a chair 304. Hence, the physical design of a wellness device could include a component of a structure that is not handheld and/or the functions of the wellness device could be embedded in any structure. Accordingly, the wellness device could have any self-contained physical design with any physical shape or size and could be a component of a larger physical structure. In the illustrated example, the back of the chair 304 could detect the breathing pattern of a person sitting on the chair 304, and render physically perceptible output based on the detected breathing pattern to sooth the user.

FIG. 3C illustrates a wellness device embodied as a stuffed animal 306. The stuffed animal 306 can detect a user's physiological activity when the user is in contact with the stuffed animal 306, or when the user is proximate to the stuffed animal 306. As shown, for example, the stuffed animal 306 can render physical outputs such as audible barking sounds or wag its tail based on detected physiological activity.

Lastly, FIG. 3D illustrates a wellness device embodied as a necklace 308. The necklace 308 can detect physiological activity of the user wearing the necklace 308 and render physically perceptible outputs based on the detected physiological activity. For example, the necklace 308 can produce an illumination pattern that tracks the detected heartbeat rate of the user wearing the necklace or render the physiological activity of any other person. Accordingly, embodiments of the wellness device include any physical design such as objects with a natural or artificial appearance, and/or embed the functions of a wellness device in other objects to facilitate widespread adoption by a broad population that could benefit from the wellness features described herein.

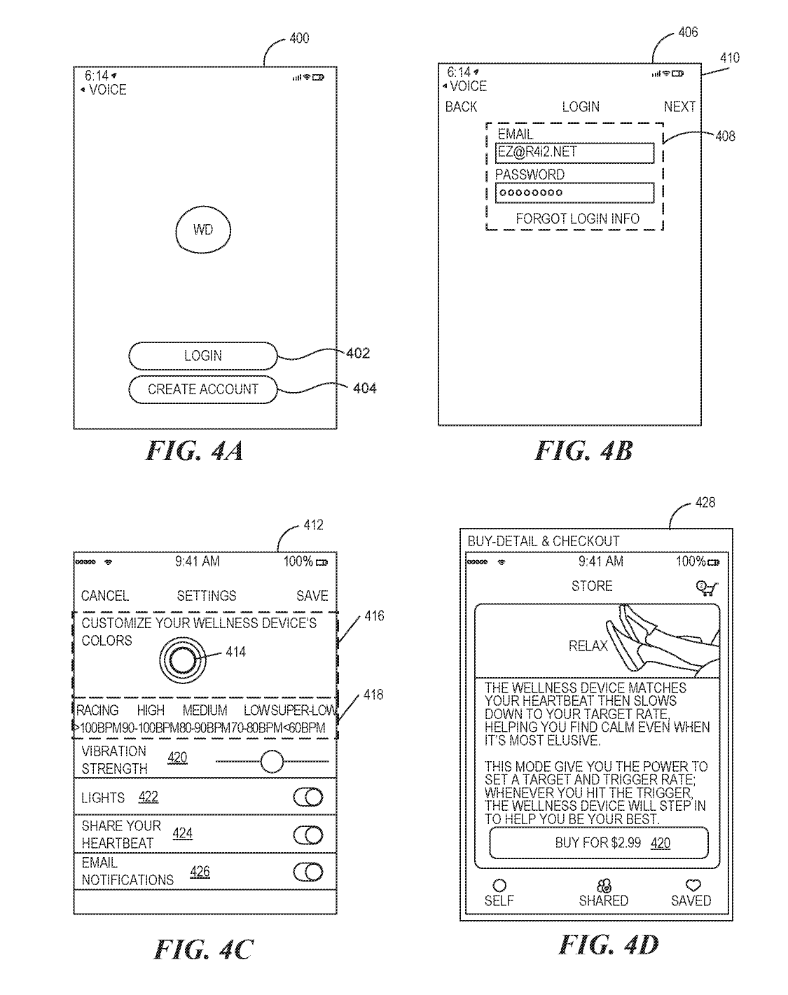

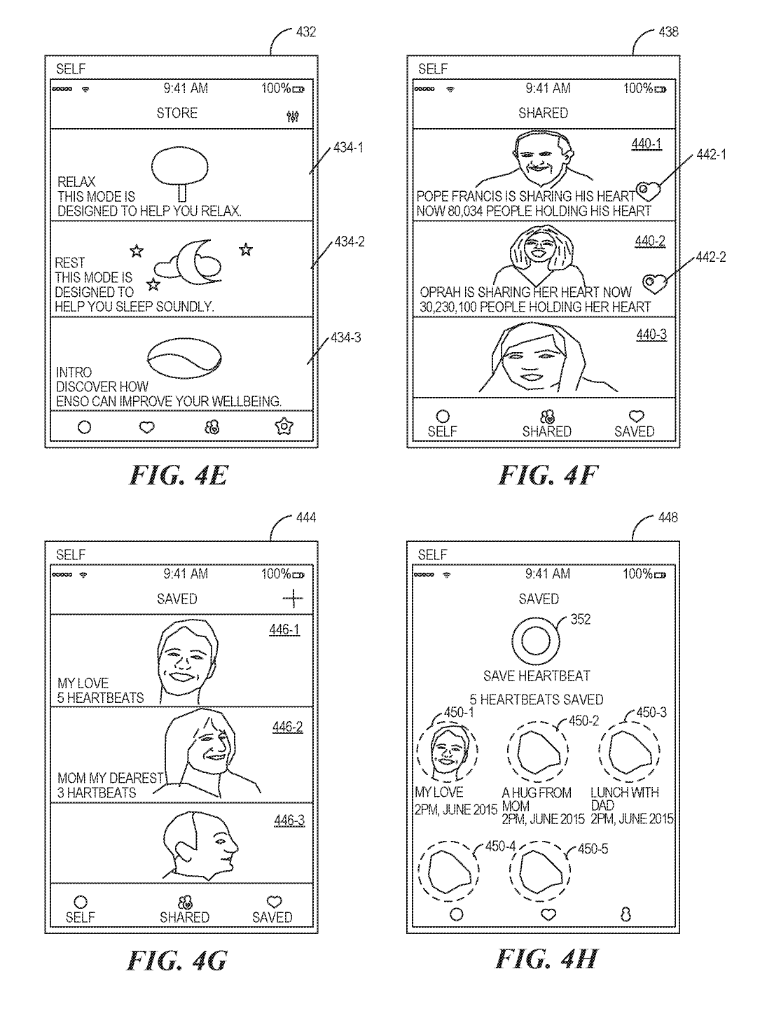

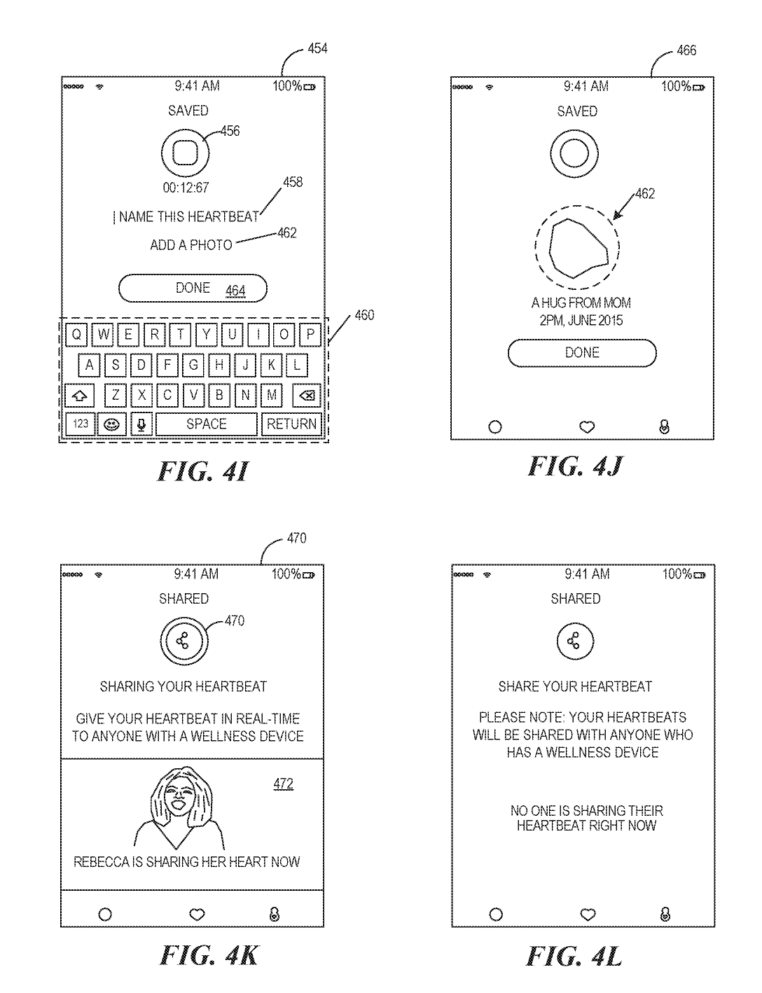

The disclosed embodiments can include a software application for a computing device used to setup and operate a wellness device. For example, FIGS. 4A through 4J illustrate various screenshots of an application (“app”) operating on a computing device, which can be communicatively coupled to the wellness device. The combination of the wellness device and the application running on the computing device can collectively form at least a portion of a wellness platform. In some embodiments, the computing device is a handheld device such as a smartphone and the application is a mobile app running on the smartphone. However, the disclosure is not limited to this particular example. For example, the computing device can be a desktop or laptop computer, or any other type of computing device operable to run the application and connect to the wellness device over a wireless or wired medium to communicate information.

FIG. 4A illustrates a screenshot of a login screen 400 of a mobile app running on a mobile device according to some embodiments of the present disclosure. As shown, the login screen 400 includes selectable controls such as a “Login” control 402 and a “Create Account” control 404. In some embodiments, selecting the “Login” control 402 causes the app to automatically open a user portal or to prompt the user for credentials required to access the user portal.

FIG. 4B illustrates a screenshot of a signup screen 406 of the mobile app according to some embodiments of the present disclosure. The signup screen 406 is displayed when a user selects the “Create Account” control 404 shown in FIG. 4A. The signup screen displays various fields 408 that identify the user and code information associated with a wellness device. Examples of the fields include “Full Name,” “Email,” “Number,” and “Password.” The signup screen 406 also includes a selectable “Next” control 410 that can be clicked to create a user account based on the information entered by the user into the fields 408 of the signup screen 406.

FIG. 4C illustrates a screenshot of a settings screen 412 of the mobile app according to some embodiments of the present disclosure. The settings screen 412 includes options to personalize outputs of a wellness device. A portion of the settings screen includes a circular cursor 414 that overlays a color pane 416. A user can use a touch input to drag the cursor 414 over a desired color in the color pane 416. The color encircled by the cursor 414 sets the color (or colors) that will be output by the wellness device. A color can be set for different heart rates 418. For example, different colors can be set for a racing heart rate (>100 bpm), high heat rate (90-100 bpm), medium heart rate (80-90 bpm), low heart rate (70-80 bpm), and super-low (<60 bpm).

The settings screen 412 includes a vibration slider control 420 that can be used to set or change the intensity of the vibrations output by the wellness device. The settings screen 412 includes a light switch control 422 to activate or deactivate light outputs. The settings screen 412 also includes a share switch control 424 that allows users to share their heartbeats, for example, in real-time. Further, the settings screen 412 also includes a an email switch control 426 to send email notifications about of the heartbeat data. The settings set by a user on the settings screen 412 can then be communicated to the wellness device over, for example, a Bluetooth wireless link, to configure the outputs of the wellness device.

FIG. 4D illustrates a screenshot of a “buy” screen 428 of the mobile app that enables a user to purchase a mode of operation profile according to some embodiments of the present disclosure. As shown, a “relax” mode is offered for purchase by the user of the mobile device. The relax mode can be downloaded by clicking the “Buy for $2.99” control 430 to cause the mobile device to download instructions over a network such as the Internet, and then communicated to the wellness device over another communications link. The wellness device can be configured to operate in accordance with the relax mode to, for example, generate outputs that match the user's heart rate and then slowdown the outputs to a target rate, or to train the user to change the user's heartbeat rate. As such, the wellness device can operate as a therapeutic device to manage the user's physiological activity.

FIG. 4E illustrates a screenshot of a “self” screen 432 of the mobile app that enables a user to manage the user's physiological information according to some embodiments of the present disclosure. In some embodiments, a user's profile may include data recorded of the user's heart rate. The self screen includes control 434-1 through 434-3 to access different modes (e.g., relax, rest, or intro modes) for the user.

FIG. 4F illustrates a screenshot of a “shared” screen 438 of the mobile app that enables a user to access publicly available shared physiological information according to some embodiments of the present disclosure. As shown, the shared screen 438 includes controls 440 to access publicly available physiological activity data of celebrities such as the Pope, Oprah Winfrey, or Malala Yousafzai. In some embodiments, the controls 440 could include a status indicator 442 of the physiological information of a person that may be sharing heartbeat in real-time or has recently stored a heartbeat in, for example, a publicly available cloud-storage. The user of the mobile device can select a person's physiological information (e.g., heartbeat data) for download or streaming, and then upload physiological information to a cloud-based storage to generate output by the wellness device. As a result, the user of the wellness device can experience a rendering of the physiological activity of a celebrity or any other person that uses their own wellness device to record data indicative of the physiological activity.

FIG. 4G illustrates a screenshot of a “saved” screen 444 of the mobile app of saved physiological information according to some embodiments of the present disclosure. The saved screen 444 includes selectable controls 446 to access, for example, heartbeat data of various loved ones or friends if the user is authorized to do so. The heartbeat data may be stored locally in a memory of the wellness device, in a memory of a local computing device that can be coupled to the wellness device, or in a cloud-based storage that can be accessible over a computer network. The selected heartbeat data can be uploaded to the wellness device such that the user can experience a rendering of the selected heartbeat data with multisensory outputs.

FIG. 4H illustrates a screenshot of a “detailed save” screen 448 of the mobile app that enables a user to access particular physiological information associated with events for a particular person according to some embodiments of the present disclosure. For example, the user can select a control from the detailed save screen 448 to access heartbeat information 450 of a person recorded during a particular event at a particular time and/or location. The selected heartbeat information 450 can be uploaded to the wellness device such that the user can experience renderings of a selected heartbeat information of a person associated with a selected event. The detailed save screen 448 also include a save control 452 that can be selected to save a new record for particular physiological information.

FIG. 4I illustrates a screenshot of a “detailed add” screen 454 for saving a physiological information according to some embodiments of the present disclosure. The detailed-add screen 454 can be opened in response to clicking the save control 452 shown in FIG. 4H. The detailed-add screen 454 includes a save control 456 which can be activated for a period of time while data of physiological activity is being recorded. The detailed add screen 454 includes fields (e.g., field 458) for entering information for the physiological activity being recorded, by using the virtual keyboard 460, and an image 462 associated with the recording. The detailed add screen 454 includes a done control 464 for saving the recording associated with the field 458 and image 462 provided by the user. For example, FIG. 4J shows a screenshot of a “saved” screen 466 for a saved physiological information including the data 468 entered in the add screen 454 according to some embodiments of the present disclosure.

FIG. 4K illustrates a screenshot of a “shared” screen 470 through which a user can share saved physiological information or stream physiological information in real-time over a communications link to another wellness device. The control 470 that can be selected to cause the mobile phone to share a physiological information collected by the user's wellness device to another user's wellness device. The shared screen 470 also includes another control 472 that can be selected to cause the mobile device to enable the user's wellness device to receive physiological information being shared by another user. As such, the sharing of physiological information can be bi-directional. In contrast, FIG. 4L illustrates a screenshot of the shared screen 470 where no other user is currently sharing physiological information. As such, the user can only share physiological information but not receive any shared physiological information of another user.

The shared physiological information may be communicated by locally paired wellness devices (e.g., over a Bluetooth connection), over a local or wide area computer network that has wellness devices connected thereto, or combinations thereof, and via different platforms such as a social media platform that could stream the physiological information of top users (e.g. celebrities).

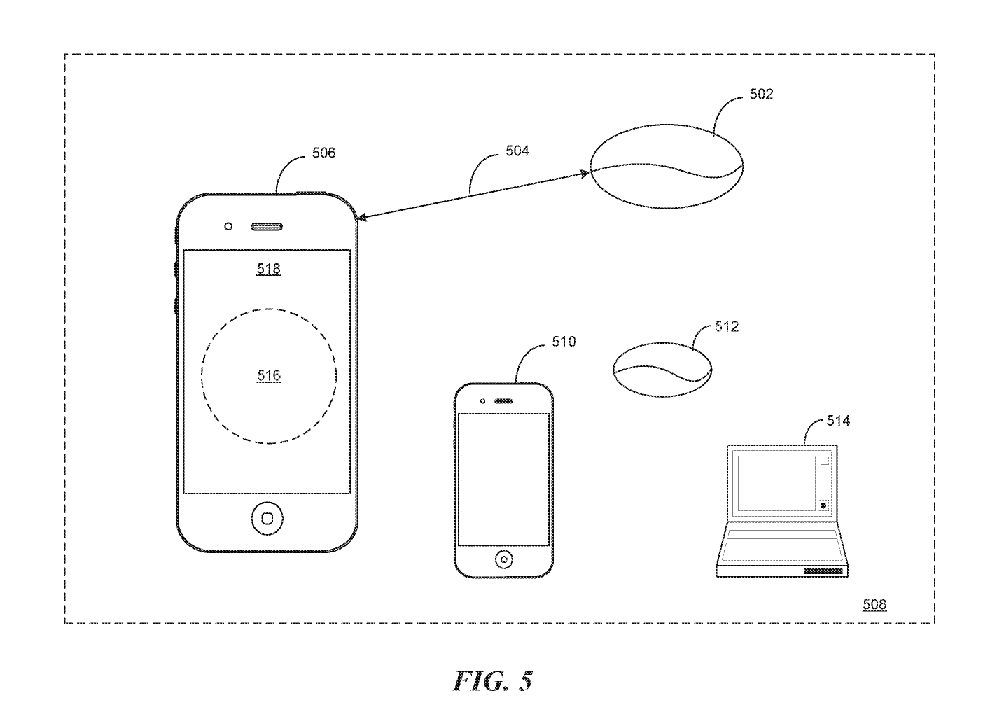

As previously discussed, a wellness device can include a wireless transceiver through which to communicate wirelessly with other wellness devices and/or other computing devices (e.g., smartphones, tablets, laptop computers). For example, FIG. 5 shows a diagram of an example wellness device 502 with a wireless communication link 504 to a computing device 506 such as a smartphone. The wireless communication link 504 may be over one more computer networks (e.g., via WiFi) or may be via a local wireless communication protocol (e.g., Bluetooth). Due to the nature of the wellness device 502, a technique for easily and wirelessly bonding the wellness device 502 with another device such as the computing device 506 is desirable.

In this context, “pairing” allows two devices to communicate data with one another, generally referring to establishing a wireless communications link between two or more devices, for the purposes of exchanging data. “Bonding” allows encrypted messages to be exchanged and, in bonding the devices, a device can store and use long term security keys. Consider, for example, a scenario in which the wellness device 502 is located at a particular physical location 508 (as indicated by the dotted line box in FIG. 5). A number of other computing devices 510, 512, and 514 may also be present at the physical location 508. Bonding the wellness device 502 with a particular computing device 506 and not the other computing devices 510, 512, or 514 presents a challenge. One solution may be to enter a particular identifier associated with the wellness device 502 via an interface of the particular computing device 506, for example through a traditional Bluetooth pairing process. While effective to an extent, such a solution may not be desirable as it adds complexity to the bonding process. Described below are techniques for wirelessly bonding the wellness device 502 with another device that utilize the characteristic sensory and tactile feedback capabilities of the wellness device 502.

In a first example bonding method, the wellness device 502 is configured to receive a bonding code from a particular other device after the pairing process has begun. For example, in an embodiment, an application of computing device 506 may cause display of a defined region 516 via a display 518 associated with the device 506. To initiate the bonding process, a user of wellness device 502 places the wellness device 502 against or in close proximity to the display 518 of the computing device 506 within the defined region 516. When the computing device 512 detects that an object is placed within the defined region (e.g., through a capacitive touch based interface) the computing device 506 begins pulsing a pre-defined pattern, for example, by changing lights on its screen. This predefined pattern may be anything, however in this context it is preferable to set a pattern that would not be confused with a human heartbeat. For example, the computing device 506 may pulse at 40 beats per minute, which would not be confused with a human heartbeat. The computing device 506 may also be programmed so that it constantly displays the predefined pattern while on the bonding screen, without needing to detect that the wellness device 502 has been placed within a defined region.

In response to detecting this predefined pulsing pattern (e.g. via measuring the light reflected from the screen of the computing device 506 via the wellness device 502's photodiode, which is part of a heartbeat sensor), the wellness device 502 may begin advertising its presence in order to pair with the computing device 506. For example, in an embodiment, the wellness device 502 includes a Bluetooth transceiver. In response to detecting the pulsing from the computing device 506, the wellness device 502 may embed the detected pattern (or a code or some other information associated with the detected pattern) in advertising packets that are then broadcast wirelessly to be picked up by surrounding devices within range of the Bluetooth signal.

While in an advertising mode, the wellness device 502 may transmit these advertising packets periodically on one or more advertising channels. The computing devices 510, 512, and 514 are looking to bond with the wellness device 502 may listen for the transmitted advertising packets via the advertising channels. Provided they are within range of the signal transmitting the advertising packets, each of the devices 510, 512, and 514 may receive the advertising packets. However, in order to complete the bonding process, the devices 510, 512, and 514 could inspect the advertising packets for the particular predefined pulsing pattern (or the code or some other information associated with the pattern). Since the particular computing device 506 generated the predefined pulsing pattern, only it will have the knowledge to complete the bonding process.

In certain situations, multiple wellness devices may be present at the physical location 508. For example, FIG. 5 shows multiple another wellness devices 512 at the physical location 508. Further, the multiple other wellness devices 502 and/or 512 may have interacted with the computing device 506 in a similar manner to wellness device 502 and may themselves be broadcasting similar advertising packets looking to bond with computing device 506. Faced with receiving advertising packets including the necessary code, the computing device 506 must resolve a correct device to bond with.

In some embodiments, the computing device 506 may complete the bonding process with an advertising wellness device that is closest in proximity. For example, if each of wellness devices 502 and 512 are transmitting advertisement packets with the correct pulse code, the computing device 506 will compare the received signals and bond with the wellness device that is closest in proximity. For example, in an embodiment, an RF receiver of the computing device 506 may generate a received signal strength indication (RSSI) that is an indication of a power level of an RF signal being received by an associated antenna. Given similar RF transmitters at the wellness devices 502 and 512, this RSSI will tend to correlate with the relative distances between a receiving antenna (i.e., the computing device 506) and each transmitting device. It should be appreciated that other means for determining relative distances between devices may also be employed. For example, in some embodiments, the computing device 506 may include other sensing means such as a camera or proximity sensor through which distances to other objects may be determined.

In some embodiments, the computing device 506 may complete the bonding process with an advertising device based on the relative timing of the transmissions. For example, the computing device 506 may complete the bonding process with an advertising device that began advertising the correct pulsing code first in time. Alternatively, the computing device 506 may complete the bonding process with an advertising device that began advertising the correct pulsing code most recently.

In some embodiments, the computing device 506 may complete the bonding process with an advertising device in response to detecting another input. In other words, the computing device 506 may complete the bonding process with an advertising device in response to detecting the combination of the correct pulsing code and the other input. For example, a user at the wellness device 502 may provide a secondary input (other than placing the device within region 516) indicating an intent to bond the wellness device 502 with the computing device 506.

This secondary input by the user may take different forms. For example, the secondary input may be a particular gesture (e.g. a sweeping motion or shaking) detected at the wellness device 502 by an accelerometer or some other motion detecting means. Similarly, in other embodiments, the secondary input may be based on a user applying pressure to the wellness device 502, inputting an audio command to the wellness device 502, or simply pressing an input button on the wellness device 502. For example, in an embodiment the wellness device 502 may include a capacitive surface (e.g. similar to a touch screen display) through a user may provide input.

Regardless of how the secondary input is received at the wellness device 502, the input itself or information indicative of the input, may be transmitted to the computing device 506 several different ways. For example, data associated with the secondary input may be included in the advertising packet along with data associated with the predefined pulse code. Alternatively, data associated with the secondary input may be transmitted via a different communication channel. For example, in response to receiving the secondary input, the wellness device 502 may emit light or an audio signal (human perceptible or not) that may be picked-up by an optical or audio sensor associated with the computing device 506.

In a second example bonding method, the wellness device 502 may be set to a discovery mode and continuously seek other devices with which to pair. Instead of the computing device 506 seeking to pair with a wellness device 502, the wellness device 502 scans for incoming signals and builds a list of available devices with which to pair. In such an embodiment, the wellness device 502 may be configured to pair with every other device that is available for pairing or, in another embodiment, with other devices within a zone defined by the bounds of received signal strength indication (RSSI).

In an embodiment, the wellness device 502 may pair with each available computing device sequentially, one after the other. While the wellness device 502 is paired with a given computing device (e.g., computing device 506), they exchange information. In an embodiment, the computing device 506 is programmed to look for a particular bonding code in the wellness device's 502 advertising packet in order to complete the bonding process. In an embodiment, a user wishing to bond computing device 506 with the wellness device 502 may open an application at the computing device 506. This application may automatically cause the computing device 506 to output a predefined bonding code via a means recognizable by the wellness device 502. For example, in an embodiment, the application may cause the computing device 506 to output a light signal (e.g. via a camera flash, the display, or some other light emitting device), in which the light may be pulsed in a predetermined pattern indicative of a code. For example, in an embodiment, the display 518 of the computing device 506 may blink alternating colors. The computing device's 506 light signal is recognizable by the wellness device 502 as a code via, for instance, the wellness device's 502 photodiode sensor. The wellness device 502 embeds the recognized bonding code in its advertising packet. In an embodiment, the wellness device 502 may transmit this bonding code to the computing device 506 via a communication protocol (e.g., Bluetooth) to allow the computing device 506 to accept or reject bonding with the wellness device 502.

As with the first example bonding method, the second example bonding method may similarly have the same issue of resolving conflicts between competing bonding requests. Similarly, these conflicts may be resolved based on any number of predefined rules. For example, the wellness device 502 may complete the bonding process with another device that is transmitting the correct code and is in closest proximity relative to other transmitting devices. In other embodiments, the wellness device 502 may complete the bonding process with another device that began transmitting the code first in time or began transmitting most recently.

In some embodiments, the wellness device 502 may complete the bonding process with a device transmitting the correct code in response to further detecting an indication of a secondary input received via the transmitting device. For example, the secondary input may be received via the application executing at computing device 506. The secondary input may be a particular gesture (e.g. a sweeping motion or shaking) detected at the computing device 506 by an accelerometer or some other motion detecting means. Similarly, in other embodiments, the secondary input may be based on a user applying pressure to the computing device 506, inputting an audio command to the computing device 506, or simply pressing an input button at the computing device 506 (e.g. via a touch screen display 518).

Regardless of how the secondary input is received at the computing device 506, the input itself or information indicative of the input, may be transmitted to the wellness device 502 several different ways. For example, data associated with the secondary input may be included in the advertising packet along with data associated with the predefined code. Alternatively, data associated with the secondary input may be transmitted via a different communication channel. For example, in response to receiving the secondary input, the computing device 506 may emit light or an audio signal (human perceptible or not) that may be picked up by an optical or audio sensor associated with the wellness device 502.

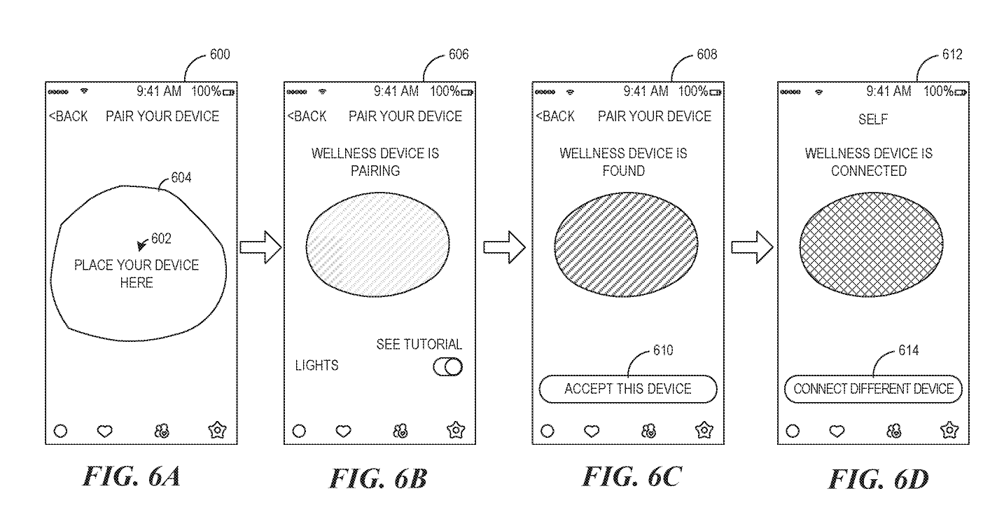

FIGS. 6A through 6D illustrate a sequence of screenshots of an application at a computing device during a process of wirelessly bonding with a wellness device, for example, as described with respect to FIG. 5.

In particular, FIG. 6A illustrates an initial screenshot 600 of the application prior to bonding with a wellness device. As shown, before bonding, the application may display a prompt 602 to the user to place a wellness device within a particular region 604 of the screen similar to that described with respect to the region 516 in FIG. 5. FIG. 6B illustrates an example screenshot 606 of the application as it begins the bonding process, for example, in response to detecting a wellness device placed in the region of the screen. FIG. 6C illustrates an example screenshot 608 of the application once the wellness device has been discovered, but before bonding is complete. As shown in FIG. 6C, the application may display a control 610 that prompts the user to accept or deny pairing with the particular discovered wellness device. FIG. 6D illustrates an example screenshot 612 of the application after bonding is complete. As shown in FIG. 6D, the application may display a control 614 that gives the user the option for the computing device to bond with another wellness device.

Embodiments of the disclosed wellness device can be configured for various use cases. For example, first responders (e.g., police, firefighters, emergency response teams) can use a wellness device as a tool to relax by feeling their own heartbeat, or a loved one's heartbeat, before and after a stressful situation. Example situations could be before the first responders go onto the shooting range for practice, before and after an emergency call (for officers in the office and out on the field).

In another example, the wellness device could be configured for at-risk youth. For example, nonprofits are bringing mindfulness and yoga activities to youth in rough neighborhoods. A wellness device could be given by those organizations as gifts to some number of the youth, to support their wellbeing. An youth can use the wellness device to feel their own heartbeat, to breathe deeply and calm down, and to keep their loved ones with them throughout the day.

Another example includes configuring the wellness device for military families. The family of a soldier deployed abroad can hold their loved one's heartbeat, in real-time, as they speak on the phone. They can even save the heartbeat of their loved one to keep with them, even though they are not in the same place.

Another example includes configuring the wellness device for therapy. Therapists can use the wellness devices to engage clients in remote therapy. For example, commands can be sent for a wellness device to trigger a “breathe” pattern, and the therapists can gauge the client's state by feeling their client's heartbeat pattern slow in real-time as they go through different therapy modalities.

Another example includes configuring a wellness device to treat post-traumatic stress disorder. For example, veterans can use wellness devices to hold a loved one's soothing heartbeat when they are reliving a traumatic moment; hence, therapists can use the wellness device to help their clients reestablish a connection with their heartbeat, breath and inner sensations.

Another example includes configuring a wellness device for long-distance couples. They can hold each other's heartbeat in real-time as they fall asleep at night. They can share their heartbeat to maintain a tangible connection from afar. Hence, they can save their partner's heartbeat to keep with them when they're far away.

Another example is configuring a wellness device for parents or children. For example, parents can save and capture the heartbeat of their child on each of their birthday's to keep forever. Another example is configuring a wellness device for palliative care. Families can capture and keep the heartbeat of a loved one who is dying. They can share their heartbeat with that loved one even when they're not in the hospital room together. Another example is configuring a wellness device for proposals. For example, people can “give their heart” to their love when they propose. Another example is configuring a wellness device as an anniversary gift. For example, married couples can give each other wellness devices to share their love. Another example is configuring a wellness device for personal journaling.

Individuals can use a wellness device to capture the feeling and energy of a particular moment in time. In another example, a wellness device could be used in meditation. That is, people can use their wellness deices as a mindfulness tool (e.g., as a point of focus). The wellness device serves to guide their breathing to a particular rhythm and re-center whenever their mind wanders.

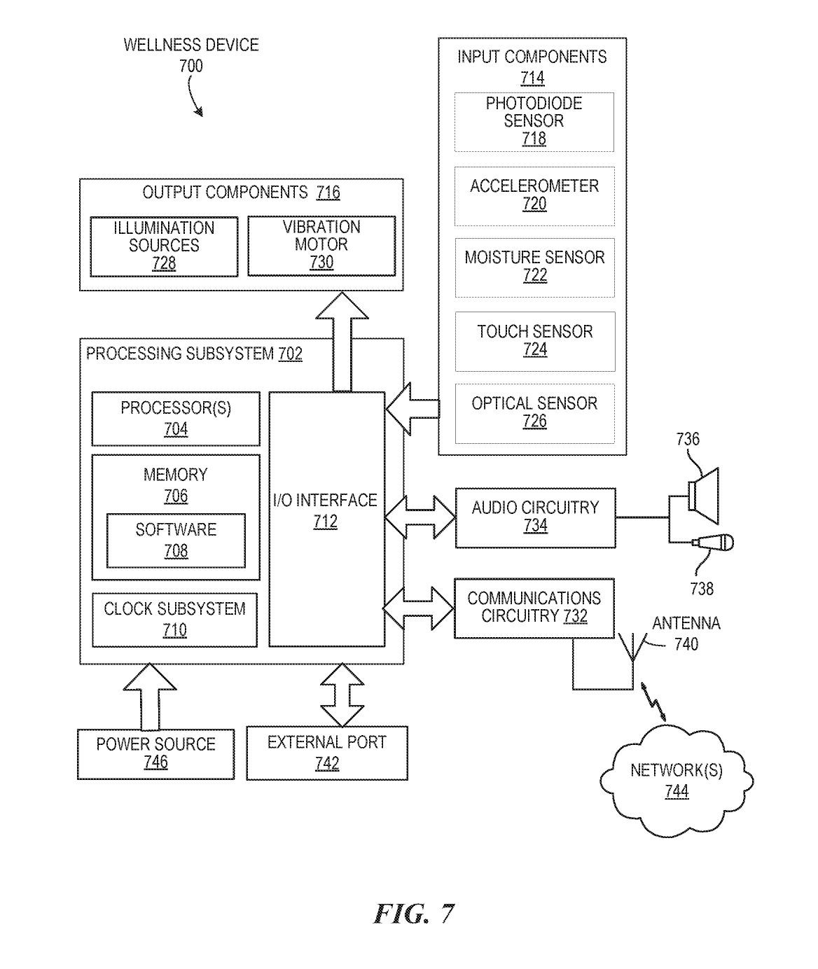

FIG. 7 is a block diagram illustrating components of a wellness device according to some embodiments of the present disclosure. The wellness device 700 may include generic components and/or components specifically designed to carry out the disclosed technology. The wellness device 700 may be a standalone computing device or part of a distributed system that spans networks, locations, and/or machines. For example, components of the wellness device 700 may be included in or coupled to a system-on-chip (SOC), a single-board computer (SBC) system, and/or other computing devices (e.g., handheld, desktop, or laptop computers). In some embodiments, the wellness device 700 can operate as a server or client device in a client-server system, or as a peer machine in a peer-to-peer system. In some embodiments, the wellness device 700 may perform processes in real-time, near real-time, and/or offline.

The wellness device 700 includes components that may be located on an electrical circuit board and interconnected over a bus (not shown) operable to transfer data between components. The wellness device 700 includes a processing subsystem 702 including one or more processor(s) 704 (e.g., Central Processing Units (CPUs), Application Specific Integrated Circuits (ASICs), and/or Field Programmable Gate Arrays (FPGAs)), a memory 706 that can store software 708, and a clock subsystem 710 that controls a timer for use in some embodiments. The memory 706 may include volatile memory (e.g., random-access memory (RAM)) and/or non-volatile memory (e.g., read-only memory (ROM)). The memory 706 may be local, remote, distributed, or combinations thereof.

The processing subsystem 702 also includes an input/output (I/O) interface 712. The components coupled to the I/O interface 712 include input components 714 and output components 716. The I/O interface 712 may include a controller operable to control the input components 714 and the output components 716. The I/O interface 712 may include other components (not shown) such as components to control physical buttons (e.g., a reset button). The input components 714 include a photodiode sensor 718 (e.g., heartbeat sensor), accelerometer 720, moisture sensor 722, touch sensor 724, and optical sensor 726. The accelerometer 720 can be operable to obtain information concerning the movement or orientation of the wellness device 700. The output components 716 include illumination sources 728 and a vibration motor 730.

Other components coupled to the I/O interface 712 include communications circuitry 732 and audio circuitry 734, including a speaker 736 and a microphone 738. The communications circuitry 732 is coupled to an antenna 740 of the wellness device 700. In some embodiments, the antenna 740 can be structurally integrated with the wellness device 700 or, for example, coupled to the wellness device 700 through an external port 742 (e.g., USB port). The communications circuitry 732 can convert electrical signals to/from electromagnetic signals that are communicated by the antenna 740 to network(s) 744 or other devices. For example, the communications circuitry 732 can include radio frequency (RF) circuitry that processes RF signals (e.g., Bluetooth signals) communicated by the antenna 740.

The communications circuitry 732 can include circuitry for performing well-known functions such as an RF transceiver, one or more amplifiers, a tuner, oscillator, a digital signal processor, a CODEC chipset, a subscriber identity module (SIM card or eSIM), and so forth. The communications circuitry 732 may communicate wirelessly via the antenna 740 with the network 744 (e.g., the Internet, an intranet and/or a wireless network, such as a cellular network, a wireless local area network (LAN) and/or a metropolitan area network (MAN)) or other devices.

In some embodiments, the antenna 740 can be programmatically controlled via the communications circuitry 732. For example, the software 708 may control or contribute to the configuration of the antenna 740 via the communications circuitry 732. For example, the memory 706 may include a database used by the software 708 to configure the communications circuitry 732 or antenna 740. The software 708 can be located anywhere in the wellness device 700 or located remotely and communicatively coupled over the network 744 via the antenna 740 to the wellness device 700. For example, the software 708 can remotely configure the communications circuitry 732 and/or the antenna 740.

The processing subsystem 702 is coupled to a power source 746. The power source 746 can include a battery. For example, a rechargeable battery can be recharged via the external port 742 when connected to another power source. In some embodiments, the power source can include a solar power source whereby the wellness device 700 includes components to collect solar power and store the power in a battery.

The software 708 can include an operating system (OS) software program, application software programs, and/or modules such as a communications module, a GPS module, and the like. For example, the GPS module can estimate the location of the wellness device 700 based on the GPS signals received by a GPS receiver (not shown) of the wellness device 700. The GPS module can provide this information to components of the wellness device 700 for use in various applications (e.g., location-based access to services).

A software program, when referred to as “implemented in a computer-readable storage medium,” includes computer-readable instructions stored in the memory (e.g., memory 706). A processor (e.g., processor(s) 704) is “configured to execute a software program” when at least one value associated with the software program is stored in a register that is readable by the processor. In some embodiments, routines executed to implement the disclosed embodiments may be implemented as part of OS software (e.g., Microsoft Windows and Linux) or a specific software application, component, program, object, module, or sequence of instructions referred to as “computer programs.”

Computer programs typically comprise one or more instructions set at various times in various memory devices of a computing device (e.g., wellness device 700), which, when read and executed by at least one processor (e.g., processor(s) 704), will cause the wellness device 700 to execute functions involving the disclosed embodiments. In some embodiments, a carrier containing the aforementioned computer program product is provided. The carrier is one of an electronic signal, an optical signal, a radio signal, or a non-transitory computer-readable storage medium (e.g., the memory 706).

Operation of a memory device (e.g., memory 706), such as a change in state from a binary one (1) to a binary zero (0) (or vice versa) may comprise a visually perceptible physical change or transformation. The transformation may comprise a physical transformation of an article to a different state or thing. For example, a change in state may involve accumulation and storage of charge or a release of stored charge. Likewise, a change of state may comprise a physical change or transformation in magnetic orientation or a physical change or transformation in molecular structure, such as a change from crystalline to amorphous or vice versa.

Aspects of the disclosed embodiments may be described in terms of algorithms and symbolic representations of operations on data bits stored in memory. These algorithmic descriptions and symbolic representations generally include a sequence of operations leading to a desired result. The operations require physical manipulations of physical quantities. Usually, though not necessarily, these quantities take the form of electric or magnetic signals that are capable of being stored, transferred, combined, compared, and otherwise manipulated. Customarily, and for convenience, these signals are referred to as bits, values, elements, symbols, characters, terms, numbers, or the like. These and similar terms are associated with physical quantities and are merely convenient labels applied to these quantities.

The wellness device 700 may include other components that are not shown nor further discussed herein for the sake of brevity. One having ordinary skill in the art will understand any hardware and software that is included but not shown in FIG. 7. While embodiments have been described in the context of fully functioning handheld devices, those skilled in the art will appreciate that the various embodiments are capable of being distributed as a program product in a variety of forms and that the disclosure applies equally, regardless of the particular type of machine or computer-readable media used to actually effect the embodiments.

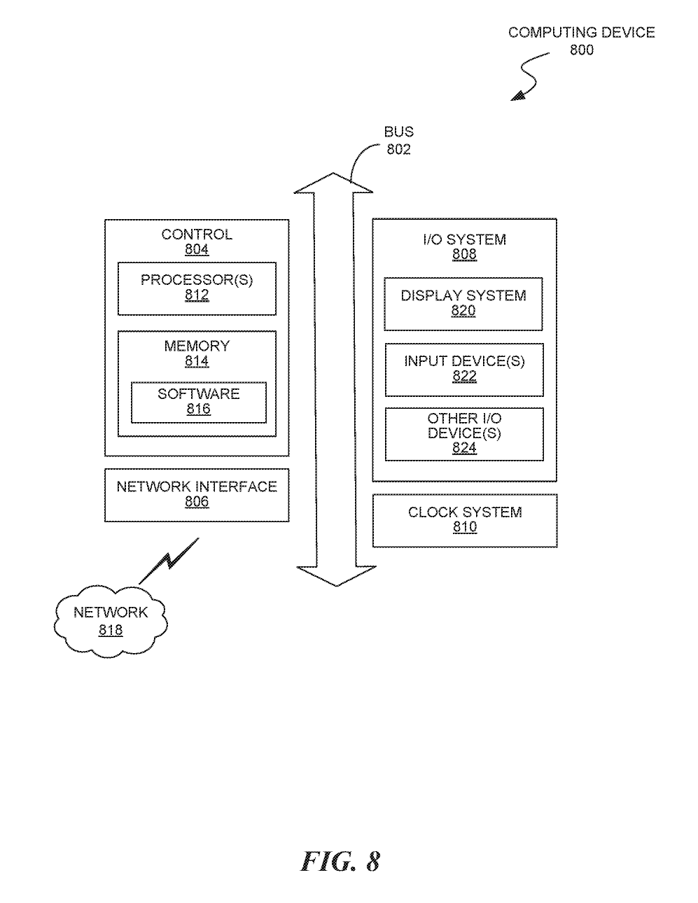

FIG. 8 is a block diagram of a computing device 800 (e.g., computing devices 506, 510, 514) operable to communicate with the wellness device 700 of FIG. 7 according to some embodiments of the present disclosure. The computing device 800 may be a generic computer or specifically designed to carry out features of a system. For example, the computing device 800 may be a SOC, a SBC system, a desktop or laptop computer, a kiosk, a mainframe, a mesh of computer systems, a handheld mobile device, or combinations thereof.

The computing device 800 may be a standalone device or part of a distributed system that spans multiple networks, locations, machines, or combinations thereof. In some embodiments, the computing device 800 operates as a server computer (e.g., serving the wellness device 700) or a client device (e.g., served by the wellness device 700) in a client-server network environment, or as a peer machine in a peer-to-peer system. In some embodiments, the computing device 800 may perform one or more processes in real-time, near real-time, offline, by batch processing, or combinations thereof.

As shown, the computing device 800 includes a bus 802 operable to transfer data between hardware components. These components include a control 804 (i.e., processing system), a network interface 806, an input/output (I/O) system 808, and a clock system 810. The computing device 800 may include other components not shown, nor further discussed for the sake of brevity. One having ordinary skill in the art will understand any hardware and software included but not shown in FIG. 8. The control 804 includes one or more processors 812 (e.g., CPUs, ASICs, FPGAs) and memory 814 (which may include software 816). The memory 814 may be local, remote, or distributed and include, for example, volatile and/or non-volatile memory.

The network interface 806 may include a modem or other interfaces (not shown) for coupling the computing device 800 to the wellness device 700 over the network 818. The I/O system 808 may operate to control various I/O devices including peripheral devices such as a display system 820 (e.g., a monitor or touch-sensitive display) and one or more input devices 822 (e.g., a keyboard and/or pointing device). Other I/O devices 824 may include, for example, a disk drive, printer, scanner, or the like. Lastly, the clock system 810 controls a timer for use by the disclosed embodiments. Many of the components of the computing device 800 operate in a manner similar to the components of the wellness device 700 and, as such, a description is omitted for the sake of brevity.

While embodiments have been described in the context of fully functioning computers, those skilled in the art will appreciate that the various embodiments are capable of being distributed as a program product in a variety of forms, and that the disclosure applies equally regardless of the particular type of machine or computer-readable media used to actually effect the distribution.

While the disclosure has been described in terms of several embodiments, those skilled in the art will recognize that the disclosure is not limited to the embodiments described herein and can be practiced with modifications and alterations within the spirit and scope of the invention. Those skilled in the art will also recognize improvements to the embodiments of the present disclosure. All such improvements are considered within the scope of the concepts disclosed herein. Thus, the description is to be regarded as illustrative instead of limiting.

1. A standalone computing device comprising:
a housing having dimensions to enable holding the standalone computing device in a palm of a user's hand, the housing including:
an input mechanism operable to detect signals indicative of the user's physiological activity obtained from the user while the user is holding the standalone computing device in the palm of the user's hand;
processing circuitry operable to generate physiological information based on the obtained signals indicative of the physiological activity, the physiological information indicative of a pattern of a bodily function of the user occurring while the user is holding the standalone computing device in the palm of the user's hand; and
an output mechanism operable to generate a physically perceptible output based on the physiological information such that the physically perceptible output is a physical rendering of the pattern of the user's bodily function.

2. The standalone computing device of claim 1, wherein the bodily function is an autonomic bodily function including any of a heart rate, a heart rate variability, a blood oxygenation level, digestion activity, a respiratory rate, a pupillary response, urination activity, or sexual arousal.

3. The standalone computing device of claim 1, wherein the bodily function is a heartbeat of the user.

4. The standalone computing device of claim 1, wherein the physically perceptible output is generated in real-time or near real-time.

5. The standalone computing device of claim 1 further comprising:
a memory operable to store the physiological information such that the physically perceptible output is based on the physiological information retrieved from the memory.

6. The standalone computing device of claim 1, wherein the physically perceptible output is synchronized with the user's bodily function.

7. The standalone computing device of claim 1 further comprising:
a memory operable to store the physiological information such that the physically perceptible output is rendered asynchronously from the stored physiological information.

8. The standalone computing device of claim 1, wherein the physically perceptible output tracks the pattern of the bodily function.

9. The standalone computing device of claim 1, wherein the physically perceptible output has at least one of a rate or magnitude.

10. The standalone computing device of claim 1, wherein the bodily function is a heartbeat of the user and the physically perceptible output includes at least one of a rate or a magnitude of the user's heartbeat.

11. The standalone computing device of claim 1, wherein the input mechanism comprises:
a sensor configured to monitor a heartbeat of the user in real-time while the user is holding the standalone computing device in the palm of the user's hand.

12. The standalone computing device of claim 1, wherein the output mechanism comprises:
a vibration motor configured to vibrate the standalone computing device in accordance with the physiological information.

13. The standalone computing device of claim 1, wherein the output mechanism comprises:
a light source configured to illuminate at least a portion of the standalone computing device in accordance with the physiological information.

14. The standalone computing device of claim 13, wherein the light source generates an illumination pattern that tracks the pattern of the bodily function.

15. The standalone computing device of claim 14, wherein the illumination pattern includes a plurality of colors.

16. The standalone computing device of claim 15, wherein each of the plurality of colors of the illumination pattern indicate a different range of the physiological information including at least one of a rate or magnitude of the pattern of the user's bodily function.

17. The standalone computing device of claim 15, where the illumination pattern changes colors in real-time or near-real time with a change of least one of a rate or magnitude of the pattern of the user's bodily function as indicated in the physiological information.

18. The standalone computing device of claim 1, wherein the standalone computing device is a first standalone computing device further comprising:
a wireless transmitter configured to transmit the physiological information to a second standalone computing device and cause the second standalone computing device to render physiological perceptible output based on the physiological information.

19. A self-contained handheld device comprising:
a body having a shape that resembles a river stone, the body including:
a sensor configured to collect signals indicative of a user's heartbeat while the self-contained handheld device is positioned on a palm of the user's hand;
a processor configured to generate physiological information that reflects a pattern of the user's heartbeat;
a vibration motor configured to cause the self-contained handheld device to vibrate in accordance with the physiological information; and
a light source configured to illuminate at least a portion of the self-contained handheld device in accordance with the physiological information.

20. A method performed by a standalone computing device, comprising:
detecting signals indicative of a user's physiological activity obtained from the user while the standalone computing device is in contact with the user's body;
generating physiological information based on the obtained signals indicative of the physiological activity, the physiological information indicative of a pattern of a bodily function of the user occurring while the standalone computing device is in contact with the user's body; and
generating a physically perceptible output based on the physiological information such that the physically perceptible output is a physical rendering of the pattern of the user's bodily function.
